(19) United States
(12) Patent Application Publication
(10) Pub. No.: US 2005/0136491 A1
(43) Pub. Date: Jun. 23, 2005

(54) Method of site specific labeling of proteins and uses therefor

(75) Inventors: Wenfang Chen, Newark, DE (US); Thomas D. Meek, Wayne, PA (US); David J. Powell, St. Albans (GB); David G. Tew, Audubon, PA (US)

Correspondence Address: GLAXOSMITHKLINE;Corporate Intellectual Property - UW2220, P.O. Box 1539, King of Prussia, PA, 19406-0939, US

(73) Assignee: SmithKline Beecham Corporation

(21) Appl. No.: 11/013,016

(22) Filed: Dec. 15, 2004

Related U.S. Application Data
(63) Continuation of application No. 09/889,344, filed on Jul. 16, 2001, now abandoned.
(60) Provisional application No. 60/117,327, filed on Jan. 22, 1999.

(51) Int. Cl. G01N 33/53; C07K 7/08; C07K 7/06

(52) U.S. Cl. 435/7.5; 530/324; 530/325; 530/326; 530/327; 530/328; 530/329; 530/330

(57) ABSTRACT

Methods for site-specific modification of protein are provided. These methods modify proteins which have been labeled at a particular site by the reaction of a transglutaminase with a glutamine peptide sequence which has been engineered into the protein. The site-specific modification methods of the invention are useful for producing reagents useful in high throughput screening methods and in producing protein delivery vehicles for specifically targeting cellular and non-cellular targets. Also described are improved biotinylation reagents.

### FIELD OF THE INVENTION

This invention relates to methods of performing bioassays, particularly high throughput screens, using site specific labeling of proteins and peptides.

### BACKGROUND OF THE INVENTION

Protein labeling methods are well known. However, these methods are often limited to the labeling of a particular protein or are cumbersome to use. It is difficult to obtain predictable labeling or to label a protein without detrimentally affecting the binding or other activity of the protein. Further, the methods are often limited to proteins which have been purified.

Genetic engineering has enabled the site specific modification of proteins. Sato et al., Biochemistry, 35, 13072-13080 (1996) describes the design of a chimeric protein of hIL-2 with a substrate sequence for transglutaminase at a terminus of the hIL-2 protein. The chimeric hIL-2 protein of Sato is then modified with two alkylamines, MDC and POE, in a reaction catalyzed by the transglutaminase.

Others have described the use of biotin, for labeling of molecules not normally biotinylated, to enable detection, purification and/or immobilization of such molecules. However, several known methods for biotinylating proteins require chemical purification of the protein. Further, methods of increasing biotin incorporation into proteins to be so labeled is desirable.

What are needed are methods for readily labeling proteins which may be in crude form.

### SUMMARY OF THE INVENTION

The present invention provides methods and reagents for performing bioassays, particularly high throughput screening wherein purification of the protein is not required.

In one aspect, the present invention provides a method of screening for a candidate compound which interacts with a first protein. The method involves modifying a first protein to contain the sequence Gln-Ser-Lys-Val-(Leu or Ile) [SEQ ID NO:1] and labeling the modified first protein by reacting a transglutaminase with the modified first protein and a detectable labeling compound. The labeled modified protein is then contacted with at least one candidate compound and the label is detected, thereby identifying the interaction of the first protein and the candidate compound.

In one embodiment, the candidate compound affects the interaction between the first protein and a second protein. In this embodiment, the method further involves the steps of contacting the labeled first protein with the second protein, and comparing binding between the labeled first protein and the second protein in the presence and absence of said candidate compound to identify a compound which affects the interaction between the first and second proteins.

In another aspect, the invention provides a method for site specific labeling of a selected protein. This method involves modifying a selected protein to contain the sequence Gln-Ser-Lys-Val-(Leu or Ile) [SEQ ID NO:1], and reacting a transglutaminase with the selected protein and a labeling compound, thereby labeling the modified protein with the labeling compound at the site of the glutamine residue.

In yet another aspect, the invention provides a modified protein labeled according to the method of the invention.

In still another aspect, the invention provides a biotinylation reagent having the formula Biotin-R¹—R², wherein X is a spacer compound and R² is a compound having at least four methylene groups and a NH₂ group. In a preferred embodiment, R¹ is selected from among Phe, Tyr, and Trp amino acids and R² is Lys.

In yet a further aspect, the invention provides a labeled modified protein useful in bioassays comprising an artificial amino acid sequence (Aa)ₙ-Gln^{P}-Ser-Lys-Val-Leu/Ile-(Aa)_{n′}, [SEQ ID NO:2], wherein n and n′ are independently selected from 0 to 100, and P is a site specific labeling compound.

The invention is advantageous in that it provides a site specific method of protein or peptide labeling wherein a first label can be incorporated into the protein and subsequently a second label can be substituted for the first label. Another advantage of the present invention is to provide a protein or peptide labeling method that can be used to monitor the expression of both soluble and insoluble proteins or “orphan” proteins. In addition, the labeling method and labeled modified protein of the invention may be readily utilized in crude protein mixtures and are thus, are particularly suitable for use in connection with automated screening methods including high throughput screens.

In still a further aspect, the invention provides a modified protein useful for targeting a moiety to a selected target, wherein the modified protein comprises an artificial amino acid sequence (Aa)ₙ-Gln-Ser-Lys-Val-Leu/Ile-(Aa)_{n′} where n and n′ are as defined above. The artificial sequence of the modified protein permits attachment of a selected moiety at a location remote from the binding site of the modified protein, thus permitting targeting of the moiety to a selected cellular or non-cellular receptor for the modified protein. The invention further provides compositions containing such a modified protein, and methods of specifically delivering a selected moiety to a target using these compositions of the invention.

Yet other advantages of the present invention will be readily apparent from the detailed description of the invention.

### BRIEF DESCRIPTION OF THE DRAWINGS

FIG. 1 is a chromatogram providing Factor XIIIa mediated labeling of C-tagged ACP with Biotin-NitroTyr-Lys-OH, at time 22 hr.

FIG. 2 is a chromatogram providing Factor XIIIa mediated labeling of C-tagged ACP with Biotin-Trp-Lys-OH, at time 22 hr.

FIG. 3 is a chromatogram 3 providing C-tagged ACP standard.

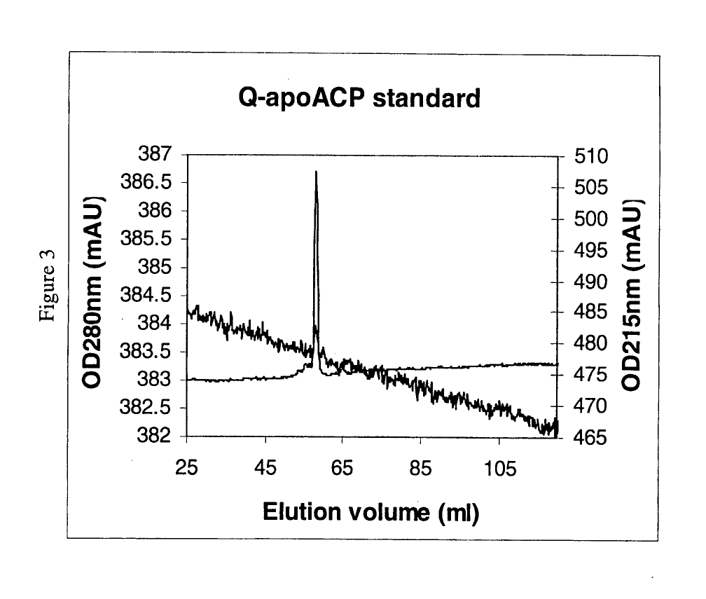

FIG. 4 is a chromatograrn providing Factor XIIIa mediated labeling of C-tagged ACP with Biotin-Trp-Lys-OH, time 22 hr, spiked with unlabeled C-tagged ACP.

### DETAILED DESCRIPTION OF THE INVENTION

In general, the present invention provides methods of site-specific labeling of a selected protein using tranglutaminase, and the use of these labeled proteins in bioassays, particularly high throughput screening assays. The labeled proteins of the invention may also be used for protein purification and immobilization. Also provided by the invention are improved biotin labels for use in these and other methods. Further provided by the invention are methods of specifically modifying a protein at a location remote from its binding site for use in specific targeting of cellular and non-cellular targets.

More particularly, the method of the invention involves modifying a protein such that it contains a defined glutamine (Gln)-containing sequence, most preferably, Gln-Ser-Lys-Val-(Leu or Ile) (hereinafter the Gin peptide sequence, SEQ ID NO:1). The modified protein is labeled by contacting it with a transglutaminase and a selected moiety which may provide a means of detecting the modified protein and/or its target (e.g., a detectable labeling compound) or another means of delivering a selected moiety to that target (e.g., a toxin).

The prior art has described the ability of transglutaminase to catalyze the reaction R—CONH₂+R′—NH_{2—}R—CONHR′+NH₃, in the presence of Ca²⁺, in which R—CONH₂ represents the acceptor, a Gln residue in proteins, and R′—NH₂, the donor, an alkylamine. However, transglutaminase does not act on every Gln residue, and the requirements for recognition of a Gln residue within a protein or peptide sequence by transglutaminase are unknown in the art.

The inventors have found that transglutaminases are able to catalyze reactions to the Gln residue, where the Gln residue is adjacent to or proximate to the above defined four amino acid sequence, regardless of the position of this peptide sequence in the protein. Thus, the method of the invention permits a label to be effectively incorporated, as desired, into any position on the protein, for example, in the N terminal region, in the C terminal region, or internally. Accordingly, a specific position can be chosen to accommodate the functional requirements of the protein. For example, it is known that N terminal modification of chemokines can affect their activity, therefore either internal or C terminal modification would be preferable. Because the method of the invention provides site specific and predictable labeling, only a single molecular species is formed. Further, since the labeling is in a predetermined position, adventitious labeling and effects on the activity of the modified protein are reduced or prevented.

Methods of Site-Specific Labeling of Proteins

Thus, in one aspect, the present invention provides a method for site specific labeling of a selected protein. Most desirably, the protein selected is of a known sequence. Alternatively, a selected protein of unknown sequence may be utilized, e.g., by fusion of the defined Gln peptide sequence to the selected protein. As used herein, the term “protein” encompasses artificial proteins, including, without limitation, fusion proteins, chimeric proteins, and the like. For convenience, “protein” will be used throughout the specification for convenience. However, it will be readily understood that a peptide sequence may be modified (or synthesized or engineered as described herein) to contain the Gln-peptide sequence defined herein and used as described for the site-specific modified proteins of the invention.

Once a suitable protein is selected, the protein is modified to contain the Gln peptide sequence as defined herein. In one preferred embodiment, the resulting modified protein contains the sequence (Aa)ₙ-Gln-Ser-Lys-Val-(Leu or Ile)-(Aa)_{n′} [SEQ ID NO:3], wherein n is from 0 to 100 [SEQ ID NO:3]. In certain embodiments, it is desirable for n to be in the range of 1 to 50, and in other embodiments it is desirable for n to be in the range of 1 to 10 or in the range of 1 to 4. This sequence may be located at the N-, or C-terminus, or imbedded within the selected protein. Thus, n′ may be independently selected from the range from 0 to 100.

The modification to the selected protein may be achieved using any suitable means, including, e.g., chemical synthesis, site specific modification of the codons encoding the amino acid sequence to be modified or other genetic engineering methods. See, generally, G. Barony and R. B. Merrifield, The Peptides: Analysis, Synthesis and Biology, Academic Press (1980); Chemical Modification of Enzymes, ed. Eyzaguirre (Ellis Horwood Limited, Chichester) (1987); Sambrook et al, Molecular Cloning: A Laboratory Manual, Cold Spring Harbor Press, Cold Spring Harbor N.Y., 1989). Alternatively, the selected protein may be modified by fusing a Gln peptide sequence (or fragment thereof required to provide the selected protein with an artificial Gln peptide sequence) to the protein by conventional means. In such a situation, the peptide sequence used for the fusion may be made by chemical synthesis or engineered using any suitable method. Where the selected protein is modified by fusing the Gln peptide sequence (or fragment thereof) to the protein, the Gln peptide sequence may be located at the N-terminus, C-terminus, or at an internal location. In one desirable embodiment, the only modification required to the selected protein is the introduction of a Gln into a suitable location in the protein (e.g., by alternation of its coding sequence).

Once the modified protein is obtained, the protein is contacted with a transglutaminase and a suitable labeling compound. The transglutaminase selected for use in the method of the invention is not a limitation of the invention, it may be readily selected by one of skill in the art. There are four known mammalian transglutaminases: plasma transglutaminase or factor XIII, tissue transglutaminase (TG_{C}), keratinocyte transglutaminase (TG_{K}) and epidermal transglutaminase (TG_{E}). Further, transglutaminases have been obtained from bacteria, including the transglutaminase from Streptoverticillium mobarense. These enzymes may be obtained from commercial sources [e.g., Sigma Chemical Co.] or isolated using techniques known to those of skill in the art. Any of these proteins or fragments thereof having native transglutaminase activity, or other selected transglutaminases, should have sufficient enzymatic activity to perform the labeling reactions described herein.

The labeling compounds useful in the invention contain a conventional detectable label linked to a compound which mimics a lysine side chain in its ability to present a primary amine for the transglutaminase catalyzed reaction and in the distance between the primary amine and the linkage with the detectable label. Such amine donor compounds may be readily selected using the guidelines provided herein. The following shows a generic transglutaminase catalyzed transamidation: 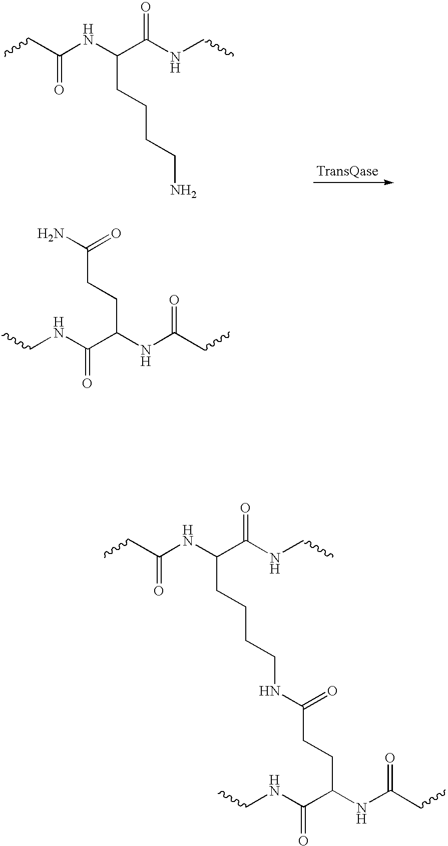
Thus, in a generic transglutaminase reaction, the lysine side chain of one (poly)peptide or protein is linked to the glutamine of a second (poly)peptide or protein. A selected amine donor compound mimics the lysine side chain by virtue of the fact that it presents the primary amine in a similar manner. The reaction for an exemplary amine donor compound, dansyl cadaverine, is shown below:

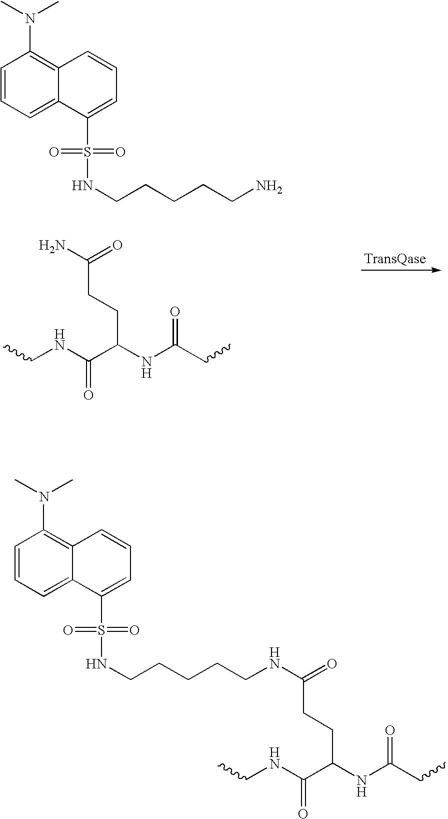

Suitable examples of such amine donor compounds include cadaverine (NH₂(CH₂)₅NH₂) and similar moieties which contains at least four methylene groups and an NH₂. These amine donor compounds are provided with conventional labels which permit their detection to form labeling compounds of the invention. Suitable detectable labels used in conjunction with the amine donor compounds may include those selected from fluorescent and non-fluorescent, radioactive, colored, substituents with latent, chemically-reactive groups (masked electrophiles or nucleophiles such as ketals, acetals, thioesters) and biotin. Some examples of labeling compounds which can be used in the method of the invention include Texas red cadaverine, tetramethyl rhodamine cadaverine, eosin cadaverine, Oregon green cadaverine, cascade blue cadaverine, bodipy TR cadaverine, fluorescein cadaverine, lucifer yellow cadaverine, rhodamine green cadaverine, and lysine derivative of a sensitizer-DTPA lanthanide chelate, and Ruthenium tris bipyridyl cadaverine. Biotin cadaverine has been found to be an acceptable labeling compound to introduce biotin. However, the inventors have designed new biotin labeling compounds which provide significantly faster and more efficient incorporation of biotin than the biotin compounds of the prior art.

Thus, in another embodiment, the present invention provides an improved labeling compound having the formula Biotin-R¹—R², wherein R¹ is a spacer compound and R² is a compound comprising at least four methylene groups and a NH₂ group. As defined herein, the spacer compound provides sufficient distance between the biotin and R², such that the spacer compound provides the resulting biotinylation reagent (labeling compound) with an ability to incorporate into the protein to be labeled which exceeds that of biotin cadaverine and other biotin molecules. Desirably, the spacer compound is a large hydrophobic compound. Suitably, such spacer compounds may be readily selected from among amino acids, including modified amino acids, and chemical compounds. In a currently preferred embodiment, R¹ is selected from among Phe, Tyr, and Trp amino acids. In another embodiment, R¹ is a naphthol group or a derivative thereof. Desirably, R² is selected from among compounds containing at least four methylene groups and NH₂. In one currently preferred embodiment, R² is lysine. However, cadaverine or other similar moieties may be readily used. The inventors have found a biotin dipeptide of the sequence Biotin-Trp-Lys-OH provides significant improvement over prior art biotin labels. These advantages are demonstrated in Example 2. Another desirable biotin dipeptide of the invention is Biotin-NitroTyr-Lys-OH. Thus, these biotin labeling compounds of the invention may be readily utilized in the methods of the invention, or for other applications for which biotin labeling is desirable.

Labeling of Proteins In Mixtures and in Solid Phase

It has surprisingly been found that the method of the invention can be used for labeling of either pure protein or crude protein mixtures in solution. The utility of this method for crude protein mixtures is unexpected since the function of transglutaminase is to cross-link proteins, e.g, fibrin cross-linking in blood clot formation. During these labeling reactions, whether in crude mixtures or with purified proteins, no non-specific cross linking is seen. Labeling of crude protein mixtures is particularly useful for use in high throughput screening methods as it reduces cost and time required for performing an assay. In particular, the ability to specifically label a labeled modified protein of the invention in crude or impure mixtures may reduce or eliminate the need for further purification prior to performing an assay. However, if additional purification of the protein is necessary, a label can be introduced into the modified protein in the crude mixture to facilitate further protein purification. Finally, labeling of a modified protein in a crude mixture also allows the expression level of the protein to be monitored.

The applicability of the method of the invention is not limited to proteins in solution. Proteins may be specifically and efficiently labeled in the solid phase, particularly when immobilized on membranes such as nitrocellulose, PVDF etc. Therefore, insoluble proteins can be detected and monitored. Further, so-called “orphan” proteins, those for which antibodies are not available, can be detected and monitored (i.e., in a manner analogous to Western blots). Additionally this allows for the detection of expression levels or changes in post translational modifications of proteins which have been appropriately engineered to contain a Gln peptide sequence. Thus all gel and membrane based techniques which require detection of proteins via an antibody can be replaced by this method without the use of antibodies. This method is therefore of great utility in proteomic analyses.

It is believed to be the amide linkage catalyzed by the transglutaminase which provides the selected modified protein of the invention with a label which is highly chemical stable yet readily removable. Both of these characteristics are significant advantages of the present invention. Without being bound by the mechanism by which the invention functions, the inventors believe that these advantages are due to the fact that the labeling compounds used are primary amines which are less reactive with modified proteins and are generally not hydrolyzed. As such, the labeling compound can be recovered in an unaltered form and reused. Further, the amide link which is formed following contacting the modified protein of the invention with transglutaminase and a first labeling compound can be replaced by an amide link of a second label which is formed using a contacting step and the second labeling compound. Thus, the first labeling compound on the modified protein may be removed by contacting the labeled modified protein with transglutaminase and the desired second labeling compound. This is particularly useful in that the modified protein of the invention may be labeled as appropriate to the specific task. For example, a modified protein can be labeled with a first labeling compound to aid in purification, such as biotin, which would provide binding of the protein to immobilized streptavidin or avidin, and then, following purification via column chromatography, the first labeling compound which permits purification may be replaced with a fluorescent labeling compound which is more appropriate for assay configuration or visualization. Furthermore, by facile substitution or replacement of the fluorescent labeling compound, the invention provides a very useful means to optimize the choice of the fluorescent labeling compound in terms of environmental effects of the modified protein-labeling compound interaction on the fluorescence of the modified protein.

Uses of Site-Specific Labeled Proteins

Thus, the invention provides a method of producing a site-specific labeled protein, having a Gln-peptide sequence as defined herein. The modified labeled protein may be readily used in a variety of applications, including bioassays, protein purification and immobilization, and for mapping protein interaction sites.

1. Bioassays

It is contemplated that the labeled modified proteins of the invention may be readily used in any bioassay. However, these methods have been found to be particularly useful for high throughput screening methods. High throughput screening methods are well known in the art and can be used to identify compounds that bind to or interact with the labeled protein. Any of the well known assay formats, for example radioimmunoassays, competitive-binding assays, Western Blot analysis, antibody sandwich assays, antibody detection, ELISA assays, fluorescence polarization, fluorescence energy transfer including fluorescence resonance energy transfer (FRET) and homogenous time-resolved fluorescence (HTRF), fluorescence intensity, fluorescence correlation spectroscopy, scintillation proximity assay (SPA), flash plate assays, and assays which require biotin incorporation to provide a recognition event for binding or immobilization of one or more components, etc. can be used. Some examples, which are intended to be illustrative and not limiting, of possible assay formats that could use transglutaminase labeled proteins or peptides are set forth below.

1) Labeling of a modified protein/peptide ligand (e.g., using a fluorescent dye) to allow monitoring of interaction with a candidate compound. As used herein, a candidate compound may be a second protein/peptide, or may be a chemical compound. Interaction between the labeled modified protein and the candidate compound may be direct, e.g., involve covalent binding or a non-covalent linkage, or may be indirect, e.g., via an intermediate compound or binding to a location in the protein or peptide which causes a conformational change to the labeled modified protein. The labeled modified protein can be in solution, bound in a vesicle or in a cell membrane, or immobilized. The interaction between the proteins may be detected by an increase in molecular mass such that the fluorescence polarization of the label can be used to monitor the interaction.

2) Labeling of a modified protein/peptide ligand (e.g., using a fluorescent dye, lanthanide chelate, radiolabel, etc.) to allow monitoring of interaction with a candidate compound which is fixed to a solid support. The interaction is then detected after separation of unbound ligand or homogeneously in the presence of unbound ligand by fluorescence intensity, radiometry, etc. as appropriate for the label incorporated.

3) Labeling of a modified protein/peptide ligand (e.g., using a fluorescent label) and labeling of a second protein/peptide with a second label such that when the two protein/peptide species interact the fluorescence intensity or lifetime of one label is modulated by the second.

4) Labeling a modified protein/peptide (e.g., using a fluorescent dye, lanthanide chelate, radiolabel, etc.) such that the action of an enzyme upon this labeled protein/peptide causes a change in the readout being used to monitor the label, e.g., protease action, to cleave the label with part of the protein/peptide. This allows the released label to be quantified either by separation or by a change in the readout appropriate to the label, a conformational change such that a property of the label is changed in a useful fashion, e.g., fluorescence intensity caused by protein quenching.

2. High Throughput Screening Assays

The transglutaminase catalyzed labeling method and the resulting labeled modified protein of the invention are particularly useful in high throughput screens and particularly in automated high throughput screening methods for the following reasons. First, the labeled protein can be used in a crude protein mixture: the protein does not need to be purified. Second, the link between the label and the protein is highly chemically stable. Third, the label can be recovered unaltered and reused. Fourth, the labeling is reversible such that a first label can easily be substituted by a second label so that the label can be adapted depending on the assay requirements. Fifth, high levels of protein labeling have been achieved.

In a preferred embodiment of automated high throughput screening, the individual sample incubation volumes are less than about 500 μl, preferably less than 250 μl, and most preferably less than about 100 μl. Such small sample volumes minimize the use of scarce candidate agents. Furthermore, the labeling methods are particularly useful in computer automated high throughput screening methods. It is contemplated that individual steps may be separately automated or that a single computer controlled robot with a single arm can perform multiple functions. In general, the assay will be configured in accordance with a standard high throughput assay format, for example using a 96, 384, or 1536 well plate, so as to screen for compounds which modulate the interaction measured in each type of assay.

3. Protein Immobilization

In addition, the methods and labeled modified proteins of the invention can be used in protein immobilization, which could be useful in protein purification via covalent column chromatography. For example, the commercially-available, chemically activatable insoluble resin, aminohexyl-Sepharose (Pharmacia), could be used with a modified protein of the invention, either in a crude mixture or in purified form, to covalently immobilize the labeled modified protein to the resin. The labeled, immobilized protein would then be readily separable from all other proteins which are not so immobilized by chromatographic methods. The covalently immobilized protein could then either be used in immobilized form, or solubilized from the resin by subsequent reaction with transglutaminase and a labeling compound. This labeling compound could include, as above, a fluorescent-cadaverine substrate, which would aid in the detection of the protein during its tranglutaminase-catalyzed detachment from the resin. Alternatively, the immobilized modified protein could be used as is in high throughput screening methods.

4. Mapping Interaction Sites

The method of the invention can be used to map interaction sites between proteins. In particular, at least two proteins, identical or non-identical, are modified and labeled using the method of the invention. Subsequently, upon specific, non-covalent association of these labeled modified proteins (for example, the formation of specific protein homo- and heterodimers and other multimers), the protein partners may then be covalently cross-linked via the proximal transglutaminase epitopes by enzymatic (transglutaminase) reaction with a ψ-diamino alkane. The covalently cross-linked protein partners are thereby detectable by protein-denaturing, analytical methods such as reverse-phase high performance liquid chromatography and sodium dodecylsulfate polyacrylamide gel electrophoresis. The modified protein of the invention is labeled in a site specific manner according to the invention and therefore, the site of the cross linking can be determined without requiring peptide mapping or protein sequencing. Moreover, a selected protein may be modified according to the invention to contain multiple Gln peptide sequences, which are located at several positions within the protein, so that the structure can be determined with respect to the second protein. This enables the interaction sites between the two proteins to be mapped.

Methods of Specifically Targeting Cellular or Non-Cellular Targets

In another aspect, the modified proteins of the invention may be used as a vehicle to deliver a selected moiety to a desired target. Advantageously, the invention permits the selected moiety to be attached to the modified protein at the site of the artificial Gln peptide sequence which is inserted at a site remote from the protein binding site. Desirably, the modified protein is capable of specifically targeting a selected host cell or binding partner. Thus, the method of the invention provides a way to modify a protein for use as a delivery vehicle without significantly interfering with its ability to specifically bind to a selected target.

In one particularly desirable embodiment, the site-specific protein modified as a delivery vehicle of the invention is an antibody, preferably a monoclonal antibody, a chimeric antibody, humanized antibody, or a functional fragment thereof, which has specificity for a selected target. Such functional fragments may encompass Fab and F(ab′)₂ fragments derived from the antibody, and synthetic molecules produced based upon the sequences of the complementarity determining regions (CDRs) of the antibodies, Fab and/or F(ab′)₂ fragments and having the same or substantially equivalent binding abilities as these antibodies or fragments. Suitable antibodies and fragments thereof may be produced using any suitable method, e.g., recombinantly, synthetically, or by a combination of these techniques. Selection of the method of production of such antibodies is not a limitation of the invention.

In another embodiment, the site-specific modified protein of the invention derived from a virus, e.g., for specifically targeting a desired cellular receptor. Alternatively, a protein derived from a cellular receptor for a specific virus (e.g., the CD4 protein) may be modified according to the invention to target a virus (e.g., for use in an anti-viral composition). Selection of the protein to be modified according to the protein invention for use as a delivery vehicle is well within the ability of one of ordinary skill in the art.

Similarly, the present invention is not limited by the selection of the moiety to be delivered by a modified protein of the invention. Such a moiety may be readily selected from among compounds which are useful for bioassay as described above, for diagnostic purposes (e.g., fluorescent dyes, radiolabels, and the like) and compounds which are useful for therapeutic purposes. Suitable therapeutic compounds include chemotherapeutic agents, e.g., toxins such as ricin, and immunotherapeutic agents, such as cytokines, interleukins, interferons, and the like. Suitable techniques, including, but not limited to protein chemistry techniques, for attachment of such moieties to the modified proteins of the invention are known in the art. See, e.g., Chemical Modification of Enzymes, ed. Eyzaguirre (Ellis Horwood Limited, Chichester) (1987) for a general discussion of protein chemistry techniques.

Thus, the method of the invention further provides methods of specifically delivering a selected moiety to a target. This method is particularly advantageous for use in vivo, where the modified proteins of the invention are prepared as a pharmaceutical composition containing an effective amount of a modified protein delivery vehicle of the invention as an active ingredient in a physiologically compatible carrier.

An aqueous suspension or solution containing the modified protein delivery vehicles (e.g., antibodies), preferably buffered at physiological pH, in a form ready for injection is preferred. The composition for parenteral administration will commonly comprise a solution of the modified protein of the invention or a cocktail thereof dissolved in an pharmaceutically acceptable carrier, preferably an aqueous carrier. A variety of aqueous carriers may be employed, e.g., 0.4% saline, 0.3% glycine, and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc.

The concentration of the modified proteins of the invention in such pharmaceutical formulation can vary widely, i.e., from less than about 0.5%, usually at or at least about 1%, to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg of a modified protein of the invention. Desirably the compositions may contain about 50 ng to about 80 mg of modified protein, or more preferably, about 5 mg to about 75 mg of modified protein according to this invention. Similarly, a pharmaceutical composition of the invention for intravenous infusion could be made up to contain about 250 ml of sterile Ringer's solution, and about 1 to about 75 and preferably 5 to about 50 mg/ml of a modified protein of the invention. Actual methods for preparing parenterally administrable compositions are well known or will be apparent to those skilled in the art. Such methods are described in more detail in, for example, Remington's Pharmaceutical Science, 15th ed., Mack Publishing Company, Easton, Pa.

It is preferred that the modified proteins of the invention, when in a pharmaceutical preparation, be present in unit dose forms. The appropriate therapeutically effective dose can be determined readily by those of skill in the art. To effectively treat an inflammatory disorder in a human or other animal, one dose of approximately 0.1 mg to approximately 20 mg per 70 kg body weight of a modified protein (e.g., a modified antibody) of this invention should be administered parenterally, preferably i.v. or i.m. (intramuscularly). Such dose may, if necessary, be repeated at appropriate time intervals selected as appropriate by a physician. Optionally, the modified proteins described herein can be lyophilized for storage and reconstituted in a suitable carrier prior to use using conventional techniques.

The present invention will now be described with reference to the following specific, non-limiting examples.

### EXAMPLE 1

Several peptide sequences that include a glutamine residue were tested as substrates for transglutaninase. The peptide sequences were based on sequences that are known to be substrates for Factor XIII, a commercially available transglutaminase [Enzyme Research Laboratories]. The following are examples of peptide sequences that were efficiently labeled; derivatives of these sequences were then engineered into proteins:

[SEQ ID NO:4]

Peptide 1:
NH₂-Leu-Ser-Leu-Ser-Gln-Ser-Lys-Val-Leu-Gly-NH₂

[SEQ ID NO:5]

Peptide 2:
NH₂-Ile-Gly-Glu-GIy-Gln-Ser-Lys-Val-Leu-Gly-NH₂

[SEQ ID NO:6]

Peptide 3:
NH₂-Leu-Gly-Pro-Gly-Gln-Ser-Lys-Val-IIe-Gly-NH₂

A variant of the above described Peptide I sequence was engineered onto the N- and C-termini of E. coli acyl carrier protein (ACP). Both engineered ACPs could be over expressed as soluble proteins in E. coli. Analysis of the overexpressed engineered ACPs showed that they were present as a mixture of apo and holo proteins.

The presence of holo ACP indicated that these engineered ACPs were biologically active with respect to endogenous E.coli phosphopantetheine transferase activity.

A variety of fluorescent and non-fluorescent cadaverines including Texas red cadaverine, tetramethyl rhodamine cadaverine, eosin cadaverine, Oregon green cadaverine, cascade blue cadaverine, bodipy TR cadaverine, fluorescein cadaverine, lucifer yellow cadaverine, rhodamine green cadaverine, and lysine derivative of a sensitizer-DTPA lanthanide chelate were successfully incorporated onto the N- and C-terminal fusions of ACP and the derivative of Peptide 1 [SEQ ID NO:4] above.

For certain cadaverine derivatives, including rhodamine green cadaverine, the efficiency of labeling of the ACP-Peptide 1 C-terminal fusion was greater than 90%. A labeling efficiency of >90% was also demonstrated when the N-terminal Peptide 1-ACP fusion was labeled with dansyl cadaverine and analysed by a combination of N-terminal sequencing and mass mapping.

The C-terminal engineered ACP (C-tagged ACP) was reacted with both biotin cadaverine and rhodamine green cadaverine in the presence of Factor XIII. Analysis of the progress of the transamidation reaction by SDS-PAGE and fluorescent imaging (rhodamine green cadaverine) or western blotting using streptavidin HRP (biotin cadaverine) showed that the engineered ACP was able to be labeled as predicted. Control experiments using native E.coli ACP lacking the engineered peptide sequence showed that these samples were not labeled. Thus the presence of the engineered Factor XIII sequence enables site specific labeling of the protein. The extent of labeling in an non-optimized reaction with rhodamine green was estimated to be greater than 85% by high-resolution ion exchange.

The specificity of Factor XIII was demonstrated by labeling crude E.coli extracts, containing expressed N-tagged and C-tagged ACPs, with rhodamine green cadaverine. SDS PAGE analysis and UV transillumination indicated that only the tagged ACPs had been labeled in each case. As described, a transglutaminase labeled protein in a crude mixture, such as a cellular extract, can be detected and monitored. Therefore, expression levels in prokaryotic and eukaryotic systems can be monitored, and recombinant proteins can be easily purified by labeling with a group amenable to purification, e.g. biotin.

The details of the experiments are provided below.

A. Peptide Labeling:

A typical peptide labeling reaction mixture contained
286 units/ml thrombin activated Factor XIIIa
1 mM peptide (i.e., Peptide 1 SEQ ID NO:4, Peptide 2 SEQ ID NO:5 or Peptide 3 SEQ ID NO:6)
0.5 mM cadaverine derivative e.g. dansyl cadaverine in a buffer of 40 mM Tris, 150 mM NaCl, 6 mM DTT, 5 mM CaCl₂, pH 8.3.

Reaction aliquots were taken out at different time points from 0 to 24 hrs. and the labeling reaction stopped by addition of EDTA to 50 mM. Samples were stored at approximately 20° C. prior to HPLC analysis. A TFA/Water/CH₃CN solvent system was used with a C18 RP-HPLC column to separate the reaction components.

The Peptide 1 [SEQ ID NO:4] substrate was labeled with both fluorescent and non-fluorescent labels: Dansyl cadaverine, rhodamine green cadaverine, fluoresceine cadaverine, and a lysine derivative of a sensitizer-DTPA lanthanide chelate.

The cadaverine derivative labeled peptides were found to elute at different percentages of acetonitrile than that of the unlabeled peptide. The labeling of the peptides was also monitored using the absorbance of the fluorescent label where possible, e.g., 502 nm for rhodamine green cadaverine. By such HPLC analyses, the estimated extent of labeling of the peptide by dansyl and rhodamine green cadaverines was greater than 90% after 24 hrs. Mass spectrometry analysis confirmed the presence of unlabelled peptide, free label and the labeled peptide in the dansyl and rhodamine green reaction mixtures.

B. Genetic Manipulations:

Four PCR oligonucleotides were designed to introduce a transglutaminase peptide tag at both the N- and C-termini of Escherichia coli Acyl Carrier Protein (ACP), which had previously been cloned into pET22(b)+[Novagen].

The oligonucleotides designed to introduce the N-terminal tag were as follows:

SEQ ID NO:7

ACP3 (5′ to 3′),:
TGT-ACC-TCA-GAC-CAT-ATG-AGC-CTG-TCC-CTG-TCC-CAG-
TCC-AAA-GTT-CTG-CCG-GGT-CCG-AGC-ACT-ATC-GAA-GAA-
CGC-GTT-AAG

SEQ ID NO:8

ACP2 (5′ to 3′),:
TGA-TGT-CAG-TCA-AGC-TTA-CGC-CTG-GTG-GCC-GTT-GAT-G

The use of this oligonucleotide pair would introduce by PCR a tag sequence (Met-Ser-Leu-Ser-Leu-Ser-Gln-Ser-Lys-Val-Leu-Pro-Gly-Pro-, SEQ ID NO:9, similar to the sequence of Peptide 1 described above) at the N-terminus of ACP, omitting the original ATG start codon of the ACP but encoding the remainder of the protein sequence. The oligonucleotides designed to introduce the C-terminal tag were as follows:

SEQ ID NO:10

ACP1 (5′ to 3′),:
TGT-ACC-TCA-GAC-CAT-ATG-AGC-ACT-ATC-GAA-GAA-CGC-G

SEQ ID NO:11

ACP4 (5′ to 3′),:
TGA-TGT-CAG-TCA-AGC-TTA-CGG-ACC-CGG-CAG-AAC-TTT-
GGA-CTG-GGA-CAG-GGA-CAG-CGC-CTG-GTG-GCC-GTT-GAT-
GTA-ATC

The use of this oligonucleotide pair would introduce by PCR a tag sequence (-Leu-Ser-Leu-Ser-Gln-Ser-Lys-Val-Leu-Pro-Gly-Pro, SEQ ID NO:12 similar to the sequence of Peptide 1 described above) at the C-terminus of ACP, introducing a new stop codon at the end of the tag. Standard PCR conditions were employed to generate each tagged ACP, using KlenTaq DNA Polymerase (Clontech Laboratories, Palo Alto, Calif.). The cycling parameters were as follows: 95° C.-5 min for 1 cycle, 95° C.-1.5 min, 55° C.-1 min, 68° C.-1 min for 30 cycles, and 68° C.-5 min for 1 cycle.

The PCR product of approximately 250 base pairs obtained in each case was restricted with Nde I and Hind III, sites which had been incorporated into the primer pairs (underlined in the primer sequences above). The amplicons were then ligated into Nde I/Hind III digested pET−22b(+) using standard cloning methodologies [See, e.g., Sambrook et al., Molecular Cloning: A Laboratory Manual, Cold Spring Harbor Press, Cold Spring Harbor N.Y., 1989)]. Inserts were confirmed by dideoxy sequencing. A single positive clone in each case of the N-tagged ACP and the C-tagged ACP were transformed into chemically competent E. coli strain LW29(DE3) [ATCC] for expression.

C. Expression of N- and C-Tagged ACPs in E.coli LW29(DE3):

1 L of terrific broth (Difco), supplemented with 100 μg/ml carbenicillin, was inoculated with a 10 ml overnight culture of LW29(DE3) cells harboring the N-tagged or C-tagged ACP pET22b(+) constructs. Cultures were grown with shaking at 37° C. to an A₆₀₀ of 1.0, when expression was induced by addition of IPTG to a final concentration of 1 mM. The cultures were grown for an additional 3 hrs; with samples being taken at times 0, 1, 2 and 3 hrs. for SDS PAGE analysis. Cells were then harvested by centrifugation. The estimated expression levels for each tagged ACP was approximately 20 mg/L.

D. Purification of N- and C-tagged ACPs:

Cell lysis was followed by a 50% isopropanol precipitation to remove contaminating E.coli proteins. The tagged ACPs were concentrated by acetic acid precipitation at pH 3.9. The redissolved ACP was applied to a Q sepharose Fast Flow column and eluted with a gradient of 50 mM Tris-HCl, pH 6.1, from 0 to 0.65 M LiCl. Each tagged ACP eluted at approximately 0.3 M LiCl. The protein was dialyzed with 40 mM Tris-HCl, pH 8.0, 150 mM NaCl. The final yield of the complete purification procedure was approximately 50%.

E. Characterization of N- and C-tagged ACPs:

Mono Q ion exchange chromatography was used to distinguish and separate the holo (phosphopantetheinated) and apo forms of the purified tagged ACPs. A gradient of 0 to 1 M NaCl in 20 mM Tris-HCl, pH 7.5 was found to give baseline separation of the two ACP species. The tagged apo ACP eluted at 0.356 M NaCl, and the tagged holo ACP eluted at 0.424 M NaCl. The apo and the holo forms of the tagged ACP eluted at lower salt concentrations than the two forms of the native ACP. FabD assays and FabH coupled assays [R. J. Heath & C. O. Rock, J. Biol. Chem., 271:10996-11000 (1996)] confirmed the biological activity of C-tagged holo ACP species.

F. Labeling of N- and C-tagged ACPs:

A 270 units/ml reaction of Factor XIII was activated by 42 units/ml immobilized thrombin in Buffer 1:40 mM Tris-HCl, pH 8.3, 0.15 M NaCl. Cadaverine derivatives (rhodamine green and biotin) were used at 0.5 mM for each labeling reaction. The labeling reaction also contained 6 mM of DTT and 5 mM CaCl₂. 0.5 mg/ml tagged ACP was used in the labeling reactions, against a 0.5 mg/ml native ACP control sample. Incubations were carried out at room temperature. Aliquots were taken from the mixtures at different time points and 50 mM EDTA was used to stop the labeling reaction (Factor XIII being Ca²⁺ dependent). NuPAGE Tris-Glycine gels (4-12%) [Novex] were used to analyze the labeling results. The rhodamine green cadaverine labeling gel was observed under UV translumination, while the biotin cadaverine labeled protein was analyzed by Western blotting/streptavidin-HRP detection. A variety of cadaverine derivatives were shown to successfully label the C-tagged ACP under the conditions described above. No label was incorporated into the native ACP control under the conditions described above.

G. Labeling of E. coli Cell Lysates Containing N-Tagged and C-tagged ACPs:

5-ml LB cultures harboring the N-tagged and C-tagged ACP constructs were induced for 2 hours with 1 mM isopropyl-1-tio-β-1-thio-β-D-galactopyranoside [IPTG]. Cells were harvested by centrifugation and lysed by sonication. The cell lysate was centrifuged further to remove the cell debris. Coomassie Plus Protein Assay Reagent (Pierce) was used to estimate the total protein concentration in the lysate supernatants (2 mg/ml in both). The N-tagged and C-tagged lysates were stored at −20° C. prior to labeling studies. 252 units/ml Factor XIIIa was added to each reaction mixture containing 1 mM rhodamine green cadaverine, 0.5 mg/ml crude protein lysate, 6 mM DTT, 5 mM CaCl₂, 0.15 M NaCl, 40 mM Tris-HCl, pH 8.3, to initiate the labeling reaction. Aliquots were taken from the reaction mixtures at times 1, 4.5, 20, and 24 hrs. 50 mM EDTA was added to each aliquot to stop the reaction. Desalting was carried out using Micro Bio-spin P6 columns [Bio-Rad] to remove the free label. The desalted samples were analyzed on the NuPAGE Tris-glycine 4-12% SDS-PAGE [Novex].

H. Detection of Tagged Proteins After Electroblotting (“Q Blotting”).

Fusions of derivatives of Peptides 1, 2 and 3 with a human chemokine (CKβ9) were prepared as described for ACP-tag constructs. E. coli crude lysates containing these fusions were fractionated by SDS-PAGE. The proteins were transferred to a nitrocellulose membrane by electroblotting. The blot was briefly rinsed with PBS-0.5% Tween-20, before incubation with a reaction mixture containing 165 units/ml Factor XIIIa, 1 mM Biotin-cadaverine, 40 mM Tris-HCl pH 8.3, 0.15 mM NaCl, 5 mM CaCl₂, 6 mM DTT. The labeling reaction was shaken at room temperature for 18 hours. The blot was then washed 5 times in excess PBS-0.05% Tween-20. The blot was incubated with 1:2000 diluted Strepavidin-HRP (Pierce) at room temperature for 45 minutes. The blot was washed extensively by shaking with PBS and PBS-0.05% Tween. Seven alternative washes of 5 minutes each were performed. Labeled protein was detected with the ECL-Plus (Amersham) detection system. All three peptide-CKβ9 fusions were shown to be labeled by this procedure. A CKβ9 only control was not labeled. This technique demonstrated the use of this technology for detection of tagged proteins on an immobile support such as nitrocellulose without the requirement of an antibody. This procedure could be used for monitoring the expression levels of a tagged recombinant protein.

### EXAMPLE 2

### Improved Biotinylation Reagents

The ACP-peptide 1 C-terminal fusion was labeled with biotin cadaverine (Molecular Probes) in a reaction mixture of 0.5 mg/ml ACP-Peptide 1 fusion, 1.5 mM biotin-cadaverine and 504 units/ml Factor XIIIa. The efficiency of incorporation was determined by competitive ELISA to be 56%.

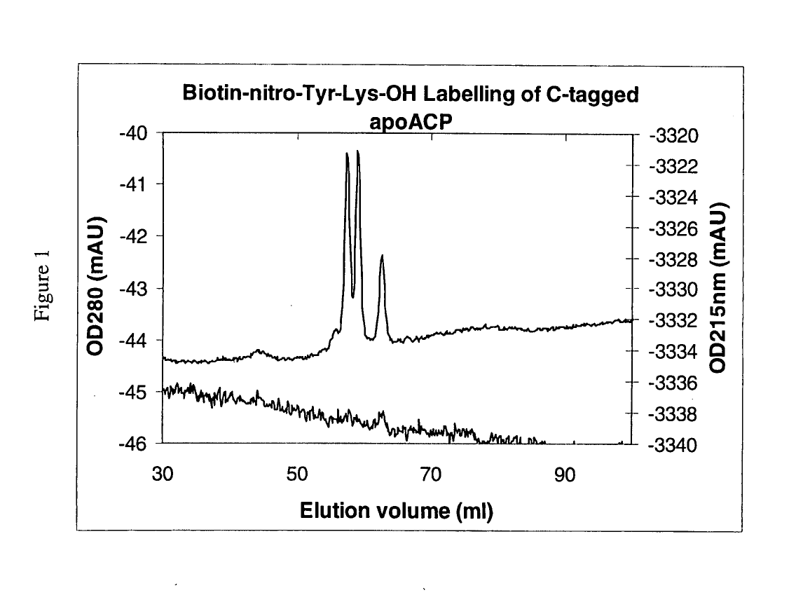

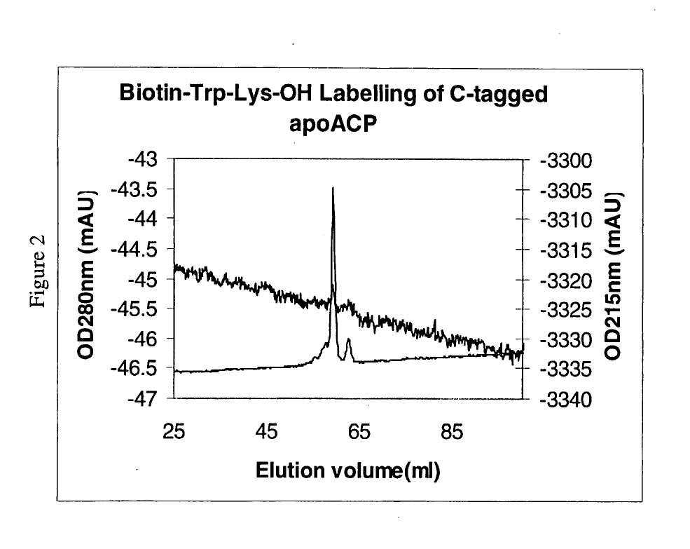

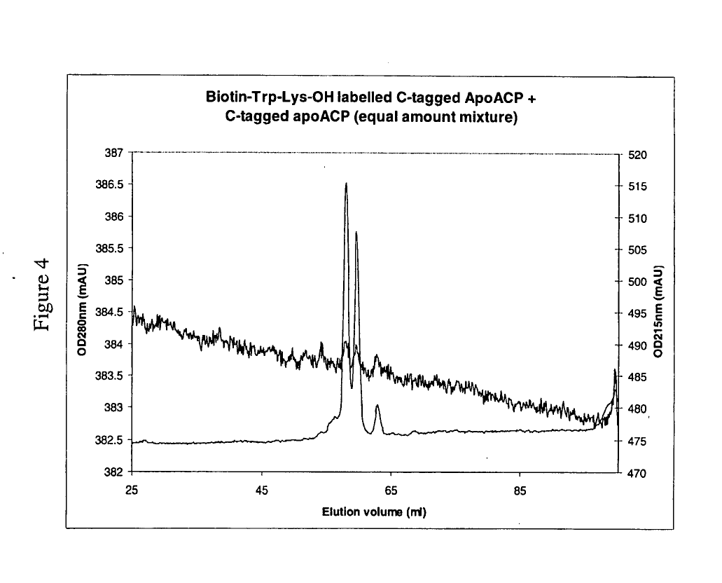

Novel biotinylation reagents (i.e. labeling compounds) were tested in an attempt to increase the yield. To this end, two biotinylated dipeptides, Biotin-Trp-Lys-OH and Biotin-NitroTyr-Lys-OH, were evaluated in a reaction mixture of 0.5 mg/ml ACP-Peptide 1 fusion, 1.5 mM biotin-cadaverine and 504 units/ml Factor XIIIa. Incorporation of the Biotin-Trp-Lys-OH dipeptide was shown by Mono Q ion exchange to be >85% (FIG. 2), in comparison to a 55% incorporation of the Biotin-NitroTyr-Lys dipeptide (FIG. 1). FIG. 2 provides the C-tagged ACP standard. The identity of the modified peak was confirmed by addition of unmodified ACP at the end of the reaction (FIG. 4).

### EXAMPLE 3

### Construction, Purification and Labeling of Q-Tagged FabH

An N-terminally Q-tagged Streptococcus haemophilus FabH gene construct was made by PCR amplification from a previously cloned FabH cDNA. The 5′ primer, SEQ ID NO:13,

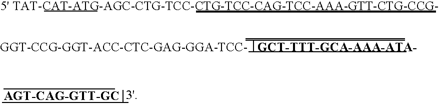

contained an NdeI restriction site (underlined) followed by the sequence encoding the Q-tag-LSLSQSKVLPGP-(SEQ ID NO:12, DNA sequence, double underline). This oligonucleotide annealed to the 5′ end of the FabH cDNA, omitting the initiating Met residue (bold, boxed DNA sequence). The 3′ primer, SEQ ID NO:14:

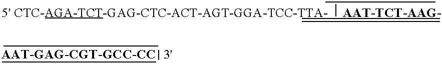

annealed to the 3′ end of the FabH gene (boxed, bold sequence) and included a stop codon (double underline). This primer contained a BglII site for cloning (underlined).

The Q-tagged FabH PCR product was amplified with Klen Taq HF polymerase (Clontech) and cloned into a T-vector (pCR2.1, Invitrogen) using standard methodologies. Following confirmation of the sequence by dideoxy sequencing, the Q-tagged FabH DNA was cloned into pET−16b [Novagen], downstream of the deca-His tag, using the NdeI and BglII restriction sites (the pET vector was digested with NdeI and BamHI, BamHI and BglII having compatable sticky ends). The protein sequence of the recombinant His-tagged, Q-tagged FabH would thus be, SEQ ID NO: 15:

MGHHHHHHHHHHSSGHIEGRHMSLSLSQSKVLPGPGTLEGSAFAKISQVA
HYVPEQVVTNHDLAQIMDTNDEWISSRTGIRQRHISRTESTSDLATEVAK
KLMAKAGITGKELDFIILATITPDSMMPSTAARVQANIGANKAFAFDLTA
ACSGFVFALSTAEKFIASGRFQKGLVIGSETLSKAVDWSDRSTAVLFGDG
AGGVLLEASEQEHFLAESLNSDGSRSECLTYGHSGLHSPFSDQESADSFL
KMDGRTVFDFAIRDVAKSIKQTIDESPIEVTDLDYLLLHQANDRILDKMA
RKIGVDRAKLPANMMEYGNTSAASIPILLSECVEQGLIPLDGSQTVLLSG
FGGGLTWGTLILTI

(the engineered Q-tag is underlined)

After confirmation of insertion, the Q-tagged FabH pET−16b construct was transformed into E. coli LW29 (DE3). A 2-litre culture of cells was induced with 1 mM IPTG and grown for 3 hours. SDS PAGE analysis of total cell extracts showed the accumulation of a protein of ˜40 kDa after induction. The anticipated size of the Q-tagged FabH was 39.2 kDa. The recombinant protein was purified to apparent homogeneity in one step by Ni-NTA chromatography [Qiagen]. Briefly, the cells were lysed into a Hepes buffer containing 5 mM imidazole. Ni-NTA resin was added and stirred gently for 2 hours. The resin was washed and bound proteins were eluted in a step batch format with increasing amounts of imidazole (to 500 mM). The Q-tagged FabH eluted at 200 mM imidazole.

The purified protein was labeled with fluorescein-cadaverine using Factor XIIIa. The reaction mixture (1 ml) contained 4 mg Q-tagged FabH, 554 units Factor XIIIa and 1.5 mM fluorescein-cadaverine. Following labeling, the reaction was fractionated by SDS PAGE and the gel subjected to UV light. The Q-tagged FabH was shown to be labeled with the fluorescein, a fluorescent band being observed at ˜40 kDa. This fluorescein labeled Q-tagged FabH protein was subsequently shown to be enzymatically active.

### EXAMPLE 4

### Construction and Labeling of a Q-Tagged Epo Receptor

A synthetic DNA fragment containing the Q-tag and IE8 epitope [residues 13-27 of a human beta amyloid peptide] was generated by sequential oligonucleotide annealing and PCR amplification. This fragment was tailed with BssHII and KpnI restriction endonuclease sites for subcloning between the same sites within the cloned Epo receptor (pmtallsEPOr) thus generating a synthetic EPO (sEPO) receptor-Q-IE8-FXa-Fc fusion protein for expression in Drosophila melanogaster cells. The resultant construct, pMtSEPOtg, was then digested with SpeI and XbaI to excise the entire EPO receptor/transglutaminase/IE8/Fc fusion. This fragment was inserted into pfastbac [Life Technologies] at the same sites for baculovirus expression, pFBEPOtg.
SEPO receptor Q-FC fusion: SEQ ID NO:16:
Underline=signal peptide
Regulator text=Epo receptor
Bold=Q tag
Italic=IE8 epitope
Bold Underline=cleavage site

Bold Double Underline=IgG FC region

MDHLGASLWPQVGSLCLLLAGAAWAPPPNLPDPKFESKAALLAARGPEEL
LCFTERLEDLVCFWEEAASAGVGPGNYSFSYQLEDEPWKLCRLHQAPTAR
GAVRFWCSLPTADTSSFVPLELRVTAASGAPRYHRVIHINEVVLLDAPVG
LVARLADESGHVVLRWLPPPETPMTSHIRYEVDVSAGNGAGSVQRVELLE
GRTECVLSNLRGRTRYTFAVRARMAEPSFGGFWSAWSEPVSLLTPSDLDP
LSLSQSKVLGVFFAEIEGR

[symbol]

[symbol]

[symbol]

[symbol]

[symbol]

[symbol]

Following baculovirus expression, the Q-tagged Epo receptor was purified to homogeneity by Protein G affinity and size exclusion chromatographies [Pharmacia]. The Q-tagged Epo receptor was labelled with rhodamine green in a reaction 277.2 units/ml of Factor XIIIa, 0.5 mg/ml EpoR and 1 mM Rhodamine green-cadaverine. A control reaction containing an EpoR species with a His tag included in place of the Q-tag was also completed. After labeling for 22 hours at room temperature the reactions were analysed by SDA PAGE/UV illumination. Only the Q-tagged EpoR species was shown to be labelled, no fluorescence was observed for the negative control.

All publications, including but not limited to patents and patent applications, cited in this specification are herein incorporated by reference as though fully set forth.

The present invention may be embodied in other specific forms without departing from the spirit or essential attributes thereof, and, accordingly, reference should be made to the appended claims, rather than to the foregoing specification, as indicating the scope of the invention.

1. A method of screening for a candidate compound which interacts with a first protein, comprising the steps of:
modifying a first protein to contain the sequence Gln-Ser-Lys-Val-(Leu or Ile), SEQ ID NO:1;
labeling said modified first protein by reacting a transglutaminase with said modified first protein and a detectable labeling compound;
contacting said labeled modified first protein with at least one candidate compound; and
detecting said label, thereby identifying the interaction of said first protein and said candidate compound.

2. The method according to claim 1, wherein said first protein is modified to comprise a sequence consisting of:
(Amino acid)ₙ-Gln-Ser-Lys-Val-(Leu or Ile)-(Amino acid)_{n′}, SEQ ID NO:3 wherein n and n′ are independently selected from 0 to 100.

3. The method according to claim 2, wherein n is 1 to 50.

4. The method according to claim 2, wherein n is 1 to 10.

5. The method according to claim 2, wherein n is 1 to 4.

6. The method according to claim 1 wherein said first protein is modified to contain the sequence by genetic engineering.

7. The method according to claim 1 wherein said first protein is modified to contain the sequence by chemical synthesis.

8. The method according to claim 1, wherein said sequence is fused to a terminus of said first protein.

9. The method according to claim 1 wherein said first protein is in a crude protein mixture.

10. The method according to claim 1 wherein at least 85% of said first protein is labeled.

11. The method according to claim 1 wherein said method further comprises the steps of:
replacing said labeling compound on said first protein with a second labeling compound.

12. The method according to claim 1 wherein said contacting step occurs in a plate comprising at least 96 wells.

13. The method according to claim 12 wherein said plate comprises 384 wells.

14. The method according to claim 12 wherein said plate comprises 1536 wells.

15. The method according to claim 1, wherein said candidate compound affects the interaction between said first protein and a second protein, said method further comprising the steps of:
contacting said labeled first protein with said second protein; and
comparing binding between said labeled first protein and said second
protein in the presence and absence of said candidate compound to identify a compound which affects the interaction between the first and second proteins.

16. The method according to claim 15, wherein said second protein is in solution.

17. The method according to claim 15, wherein said second protein is bound in a vesicle.

18. The method according to claim 15, wherein said second protein is bound in a cell membrane.

19. The method according to claim 15, wherein said second protein is immobilized.

20. The method according to claim 15, wherein said interaction is detected by an increase in molecular mass.

21. A method for site specific labeling of a selected protein comprising the steps of:
modifying a selected protein to contain a sequence comprising Gln-Ser-Lys-Val-(Leu or Ile), SEQ ID NO:1; and
reacting a transglutaminase with said selected protein and a labeling compound, thereby labeling said protein with said labeling compound at the site of said glutamine residue.

22. The method according to claim 21, wherein said modified protein contains a sequence consisting of:
(Aa)ₙ-Gln-Ser-Lys-Val-(Leu or Ile)-(Aa)_{n′}, SEQ ID NO:3, wherein n and n′ are independently selected from 0 to 100.

23. The method according to claim 21, wherein n is 1 to 50.

24. The method according to claim 21, wherein n is 1 to 10.

25. The method according to claim 21, wherein n is 1 to 4.

26. The method according to claim 21, wherein said protein is a crude protein.

27. The method according to claim 21, wherein said labeling compound is selected from the group consisting of cadaverines and biotin containing labels.

28. The method according to claim 27, wherein the labeling compound is a fluorescent cadaverine.

29. A protein labeled according to the method of claim 21.

30. A biotinylation reagent having the formula Biotin-R¹-R², wherein X is a spacer compound and R² is a compound comprising at least four methylene groups and a NH₂ group.

31. The biotinylation reagent according to claim 30, wherein R¹ is selected from the group consisting of Phe, Tyr, and Trp amino acids.

32. The biotinylation reagent according to claim 30, wherein R² is selected from the group consisting of lysine (Lys) and cadaverine.

33. The biotinylation reagent according to claim 30 consisting of Biotin-Trp-Lys-OH.

34. The biotinylation reagent according to claim 30 consisting of Biotin-NitroTyr-Lys-OH.

35. A site specific labeled protein comprising an artificial amino acid sequence:
(Aa)ₙ-Gln^{P}-Ser-Lys-Val-(Leu or Ile)-(Aa)_{n′} SEQ ID NO:3, wherein n and n′ are independently selected from 0 to 100, and P is a site specific labeling compound.

36. A molecule comprising a site specific modified protein delivery vehicle comprising an artificial amino acid sequence: (Aa)ₙ-Gln-Ser-Lys-Val-(Leu or Ile)-(Aa)_{n′} SEQ ID NO:3, wherein n and n′ are independently selected from 0 to 100, and a moiety to be delivered to a target by the modified protein delivery vehicle.

37. The molecule according to claim 36, wherein the delivery protein is selected from among antibodies and functional fragments thereof.

38. A composition comprising a molecule according to claim 36 and a physiologically compatible carrier.


SEQUENCE LISTING (16 sequences)

SEQ ID NO: 1 - protein, 5 aa, Artificial Sequence
  unsure (5): Where Xaa at position (5) can represent Leucine or Isoleucine
QSKVX

SEQ ID NO: 2 - protein, 207 aa, Artificial Sequence
  unsure (1)(2)(3)(4)(5)(6)(7)(8)(9)(10)(11)(12)(13)(14)(15)
  (16)(17)(18)(19)(20)(21)(22)(23)(24)(25)(26)(27)(28)
  (29)(30)(31)(32)(33)(34)(35)(36)(37)(38)(39)(40)(41)
  (42)(43)(44)(45)(46)(47)(48)(49)(50)(51)(52)(53)(54)
  (55)(56)(57)(58)(59)(60)(61)(62)(62)(64)(65)(66)(67)
  (68)(69)(70)(71)(72)(73)(74)(75)(76)(77)(78)(79)(80)
  (81)(82)(83)(84)(85)(86)(87)(88)(89)(90)(91)(92)(93)
  (94)(95)(96)(97)(98)(99)(100)(101)(106)(107)(108)(109)
  (110)(111)(112)(113)(114)(115)(116)(117)(118)(119)(120)
  (121)(122)(123)(124)(125)(126)(127)(128)(129)(130)(131)
  (132)(133)(134)(135)(136)(137)(138)(139)(140)(141)(142)
  (143)(144)(145)(146)(147)(148)(149)(150)(151)(152)(153)
  (154)(155)(156)(157)(158)(159)(160)(161)(162)(163)(164)
  (165)(166)(167)(168)(169)(170)(171)(172)(173)(174)(175)
  (176)(177)(178)(179)(180)(181)(182)(183)(184)(185)(186)
  (187)(188)(189)(190)(191)(192)(193)(194)(195)(196)(197)
  (198)(199)(200)(201)(202)(203)(204)(205)(206)(207): Where Xaa can represent none or any one of the twenty naturally
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXQSKVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 3 - protein, 207 aa, Artificial Sequence; identical to SEQ ID NO: 2
  unsure (1)(2)(3)(4)(5)(6)(7)(8)(9)(10)(11)(12)(13)(14)(15)
  (16)(17)(18)(19)(20)(21)(22)(23)(24)(25)(26)(27)(28)
  (29)(30)(31)(32)(33)(34)(35)(36)(37)(38)(39)(40)(41)
  (42)(43)(44)(45)(46)(47)(48)(49)(50)(51)(52)(53)(54)
  (55)(56)(57)(58)(59)(60)(61)(62)(62)(64)(65)(66)(67)
  (68)(69)(70)(71)(72)(73)(74)(75)(76)(77)(78)(79)(80)
  (81)(82)(83)(84)(85)(86)(87)(88)(89)(90)(91)(92)(93)
  (94)(95)(96)(97)(98)(99)(100)(101)(106)(107)(108)(109)
  (110)(111)(112)(113)(114)(115)(116)(117)(118)(119)(120)
  (121)(122)(123)(124)(125)(126)(127)(128)(129)(130)(131)
  (132)(133)(134)(135)(136)(137)(138)(139)(140)(141)(142)
  (143)(144)(145)(146)(147)(148)(149)(150)(151)(152)(153)
  (154)(155)(156)(157)(158)(159)(160)(161)(162)(163)(164)
  (165)(166)(167)(168)(169)(170)(171)(172)(173)(174)(175)
  (176)(177)(178)(179)(180)(181)(182)(183)(184)(185)(186)
  (187)(188)(189)(190)(191)(192)(193)(194)(195)(196)(197)
  (198)(199)(200)(201)(202)(203)(204)(205)(206)(207): Where Xaa can represent none or any one of the twenty naturally

SEQ ID NO: 4 - protein, 10 aa, Artificial Sequence
  Derivative of a factor XIII substrate
LSLSQSKVLG

SEQ ID NO: 5 - protein, 10 aa, Artificial Sequence
  Derivative of a factor XIII substrate
IGEGQSKVLG

SEQ ID NO: 6 - protein, 10 aa, Artificial Sequence
  Derivative of a factor XIII substrate
LGPGQSKVIG

SEQ ID NO: 7 - DNA, 81 bp, Unknown
  Oligonucleotide designed to introduce Q tag
tgtacctcagaccatatgagcctgtccctgtcccagtccaaagttctgccgggtccgagcactatcgaagaacgcgttaag

SEQ ID NO: 8 - DNA, 37 bp, Unknown
  Oligonucleotide designed to introduce Q tag
tgatgtcagtcaagcttacgcctggtggccgttgatg

SEQ ID NO: 9 - protein, 14 aa, Artificial Sequence
  Derivative of a factor XIII substrate
MSLSLSQSKVLPGP

SEQ ID NO: 10 - DNA, 37 bp, Unknown
  Oligonucleotide designed to introduce Q tag
tgtacctcagaccatatgagcactatcgaagaacgcg

SEQ ID NO: 11 - DNA, 78 bp, Unknown
  Oligonucleotide designed to introduce Q tag
tgatgtcagtcaagcttacggacccggcagaactttggactgggacagggacagcgcctggtggccgttgatgtaatc

SEQ ID NO: 12 - protein, 12 aa, Artificial Sequence
  Derivative of E. coli ACP protein
LSLSQSKVLPGP

SEQ ID NO: 13 - DNA, 92 bp, Unknown
  Oligonucleotide designed to introduce Q tag into Streptococcus haemophilus FabH gene
tatcatatgagcctgtccctgtcccagtccaaagttctgccgggtccgggtaccctcgagggatccgcttttgcaaaaataagtcaggttgc

SEQ ID NO: 14 - DNA, 53 bp, Unknown
  Oligonucleotide designed to introduce Q tag into Streptococcus haemophilus FabH gene
ctcagatctgagctcactagtggatccttaaattgtaagaatgagcgtgcccc

SEQ ID NO: 15 - protein, 364 aa, Artificial Sequence
  Modified sequence of Streptococus haemophilus FabH
MGHHHHHHHHHHSSGHIEGRHMSLSLSQSKVLPGPGTLEGSAFAKISQVAHYVPEQVVTNHDLAQIMDTNDEWISSRTGIRQRHISRTESTSDLATEVAKKLMAKAGITGKELDFIILATITPDSMMPSTAARVQANIGANKAFAFDLTAACSGFVFALSTAEKFIASGRFQKGLVIGSETLSKAVDWSDRSTAVLFGDGAGGVLLEASEQEHFLAESLNSDGSRSECLTYGHSGLHSPFSDQESADSFLKMDGRTVFDFAIRDVAKSIKQTIDESPIEVTDLDYLLLHQANDRILDKMARKIGVDRAKLPANMMEYGNTSAASIPILLSECVEQGLIPLDGSQTVLLSGFGGGLTWGTLILTI

SEQ ID NO: 16 - protein, 503 aa, Artificial Sequence [sequence omitted]